SEQUENCE LISTING (11 sequences)

SEQ ID NO: 1 - protein, 5 aa, Artificial Sequence
  Linker 1
GGGGS

SEQ ID NO: 2 - protein, 5 aa, Artificial Sequence
  Linker 2
SGGGG

SEQ ID NO: 3 - protein, 5 aa, Artificial Sequence
  Linker 3
SRSSG

SEQ ID NO: 4 - protein, 5 aa, Artificial Sequence
  Linker 4
SGSSC

SEQ ID NO: 5 - protein, 12 aa, Artificial Sequence
  Linker 5
GKSSGSGSESKS

SEQ ID NO: 6 - protein, 6 aa, Artificial Sequence
  Linker 6
RPPPPC

SEQ ID NO: 7 - protein, 7 aa, Artificial Sequence
  Linker 7
SSPPPPC

SEQ ID NO: 8 - protein, 14 aa, Artificial Sequence
  Linker 8
GSTSGSGKSSEGKG

SEQ ID NO: 9 - protein, 18 aa, Artificial Sequence
  Linker 9
GSTSGSGKSSEGSGSTKG

SEQ ID NO: 10 - protein, 18 aa, Artificial Sequence
  Linker 10
GSTSGSGKPGSGEGSTKG

SEQ ID NO: 11 - protein, 14 aa, Artificial Sequence
  Linker 11
EGKSSGSGSESKEF

(19) United States
(12) Patent Application Publication
(10) Pub. No.: US 2021/0206821 A1
(43) Pub. Date: Jul. 8, 2021

(54) CYTOKINE FUSION POLYPEPTIDE AND CYTOKINE LIBRARY COMPRISING SAME

(71) Applicant: Daegu Gyeongbuk Institute of Science and Technology, Daegu (KR)

(72) Inventors: Kyung Moo YEA, Daegu (KR); Min Seok KIM, Daegu (KR)

(21) Appl. No.: 17/059,513

(22) PCT Filed: Jun. 24, 2019
(86) PCT No.: PCT/KR2019/007602

§ 371 Date: Nov. 30, 2020

(30) Foreign Application Priority Data
Jun. 22, 2018 (KR) 10-2018-0072220
Nov. 19, 2018 (KR) 10-2018-0142513

(51) Int. Cl. C07K 14/52; C07K 14/705; C12N 15/86

(52) U.S. Cl. CPC C07K 14/52; C07K 14/705; C07K 2319/03; C07K 2319/33; C12N 15/86

(57) ABSTRACT

The present invention relates to a cytokine fusion polypeptide or a cytokine library comprising the same, and according to the cytokine fusion polypeptide or a cytokine library comprising the same according to an aspect, it enables continuous stimulation by a cytokine to a target cell and thus can easily and accurately evaluate the functionality of cytokines at the single cell level.

### TECHNICAL FIELD

It relates to a cytokine fusion polypeptide and a cytokine library comprising the same.

### BACKGROUND ART

Cytokines refer to proteins, peptides and glycoproteins secreted by various types of cells, including immune cells and it regulates various biological activities such as cell activation, differentiation, cell migration, aging and death induction in various cells through autocrine and paracrine actions. Changes in cell activity caused by cytokines may be specific to cytokines by interaction with receptors on the cell surface and cell signaling resulting therefrom.

In addition, each of the receptors expressed on the surface of all cells has a specific function, and the above function is known to have a high correlation with the cell specific function. Receptors present on the surface of these cells play a role in detecting signals received from the outside, and at the same time amplifying the received signals in the form of secondary messengers, enabling cellular activities such as metabolism, secretion, release and cell growth. When a specific ligand binds to the extracellular part, specifically the part where the receptor is located on the cell membrane, the receptor is transformed in the intracellular part by interaction with the ligand, and the initiation of signaling performs various functions of the cell. Cytokines, as protein mediators, can have a great influence on these biological processes. That is, cytokines are regulated by activation or inactivation of cytokine genes by specific cell signaling, which is closely involved in biological or pathological changes in cells.

Under this technical background, various studies on the functionality of cytokines related to changes in biological activity of cells are being conducted (Korean patent publication Np. 10-2013-0032606), but it is still insufficient.

### DISCLOSURE

### Technical Problem

An aspect of the present invention provides a fusion polypeptide comprising cytokines, transmembrane domain, and a linker connecting the cytokine and the transmembrane domain.

Another aspect of the present invention provides a polynucleotide encoding the fusion polypeptide.

Another aspect of the present invention provides a vector comprising the polynucleotide.

Another aspect of the present invention provides a host cell comprising the vector.

Another aspect of the present invention provides a cytokine library comprising the fusion polypeptide, the polynucleotide, the vector, or the host cell.

Another aspect of the present invention provides a method of screening cytokine comprising identifying any one change selected from the group consisting of a biological change of cell by a cytokine from the host cell, a change in the expression or activity of an exogenous or endogenous gene or protein and a combination thereof.

### Technical Solution

One aspect provides a fusion polypeptide comprising cytokines, transmembrane domain, and a linker connecting the cytokine and the transmembrane domain.

As used herein, the term “cytokine” is a signal substance that controls the defense system in the body and stimulates the living body, and is one of the physiological activity control peptides. Cytokines regulate various biological activities such as activation, growth, differentiation, migration, aging and death induction in various cells through autocrine and paracrine actions. Herein, the cytokine may be included without limitation as long as it is an in vivo peptide having the above-described properties. The cytokine may be, for example, any one or more selected from the cytokines shown in Tables 1 to 7, and, e.g. BMP (bone morphogenetic protein) family, CCL (Cheomkine ligands) family, CMTM (CKLF-like MARVEL transmembrane domain containing member) family, CXCL (C-X-C motif ligand ligand) family, GDF (growth/differentiation factor) family, growth hormone, IFN (Interferon) family, IL (Interleukin) family, TNF (tumor necrosis factors) family, or a combination thereof.

As used herein, the term “transmembrane domain” refers to a region penetrating the cell membrane of protein which breaks through the cell membrane and most of them are known to be composed of hydrophobic amino acids having an a-helix structure. The transmembrane domain immobilizes cytokines on the cell membrane or plasma membrane of the target cell, displays the cytokine bound thereto on the surface of the target cell, or binds the cytokine to the cell membrane receptor of the target cell and so that it can play a role of continuous stimulation by cytokines. The transmembrane domain may be a transmembrane domain of receptor tyrosine kinases (RTKs). More specifically, the transmembrane domain of tyrosine kinases may be transmembrane domain of any one receptor selected from the group consisting of epidermal growth factor receptor, insulin receptor, platelet-derived growth factor receptor, vascular endothelial growth factor receptor, fibroblast growth factor receptor, cholecystokinin (CCK) receptor, neurotrophic factor (NGF) receptor, hepatocyte growth factor (HGF) receptor, ephrin (Eph) receptor, angiopoietin receptor and RYK (related to receptor tyrosine kinase) receptor.

In one embodiment, the cytokine and the transmembrane domain may be linked through a linker. The linker can not only connect the cytokine and the transmembrane domain, but also play a role of exposing the cytokine to the surface of the target cell according to the fluidity of the linker. As the linker, for example, a flexible linker may be applied. In addition, the linker may have resistance to protease resistance, which may be used by appropriately changing its type and/or length according to cytokines or target cells. For example, the linker may be a polypeptide consisting of any amino acids of 1 to 400, 1 to 200, or 2 to 200. The peptide linker may include Gly, Asn and Ser residues, and neutral amino acids such as Thr and Ala may also be included. Amino acid sequences suitable for peptide linkers are known in the art. It is also possible to adjust the copy number “n” by taking into account the optimization of the linker for achieving proper separation between functional moieties or maintaining the essential inter-moiety interactions. Other flexible linkers are known in the art, for example, G and S linkers that add amino acid residues such as T and A to maintain flexibility as well as add polar amino acid residues so as to improve water solubility. Thus, in one embodiment, the linker may be a flexible linker including G, S and/or T residues. The linker may have a general formula selected from (GₚSₛ)ₙ and (SₚGₛ)ₙ, wherein, independently, p is an integer of 1 to 10, s is 0 or an integer of 0 to 10, and p+s is an integer of 20 or less, and n is an integer of 1 to 20. More specifically, examples of linkers include (GGGGS)ₙ (SEQ ID NO: 1), (SGGGG)ₙ (SEQ ID NO: 2), (SRSSG)ₙ (SEQ ID NO: 3), (SGSSC)ₙ (SEQ ID NO: 4), (GKSSGSGSESKS)ₙ (SEQ ID NO: 5), (RPPPPC) n (SEQ ID NO: 6), (SSPPPPC) n (SEQ ID NO: 7), (GSTSGSGKSSEGKG)ₙ (SEQ ID NO: 8), (GSTSGSGKSSEGSGSTKG)ₙ (SEQ ID NO: 9), (GSTSGSGKPGSGEGSTKG)ₙ (SEQ ID NO: 10) or (EGKSSGSGSESKEF)ₙ (SEQ ID NO: 11), wherein n is an integer of 1 to 20, or 1 to 10.

In addition, the cytokine fusion polypeptide can provide continuous stimulation for target cells by self-secreting cytokines to target cells. Accordingly, the transmembrane domain may penetrate and immobilize the cell membrane of the target cell, and the cytokine may bind to the cell membrane receptor of the target cell to stimulate the target cell.

Another aspect provides a polynucleotide encoding the fusion polypeptide.

As used herein, the term “polynucleotide” refers to a polymer of deoxyribonucleotides or ribonucleotides present in a single-stranded or double-stranded form. It includes RNA genomic sequences, DNA (gDNA and cDNA) and RNA sequences transcribed therefrom, and unless specifically stated otherwise, it includes natural polynucleotides as well as analogs thereof with modified sugar or base sites. In one embodiment, the polynucleotide is a single chain polynucleotide.

Another aspect provides a vector comprising the polynucleotide.

As used herein, the term “vector” is a vector capable of expressing a protein of interest in a suitable host cell, and refers to a genetic construct comprising a regulatory element operably linked to express a gene insert. A vector according to an embodiment may include an expression control element such as a promoter, an operator, an initiation codon, a stop codon, a polyadenylation signal and/or an enhancer, and the promoter of the vector may be constitutive or inducible. In addition, the vector may be an expression vector capable of stably expressing the fusion protein in a host cell. The expression vector may be a conventional one used in the art to express foreign proteins in plants, animals or microorganisms. The recombinant vector can be constructed through various methods known in the art. For example, the vector may include a selectable marker for selecting a host cell containing the vector, and in the case of a replicable vector, it may include an origin of replication. In addition, the vector can be self-replicating or introduced into the host DNA and the vector may be selected from the group consisting of a plasmid, a lentivirus, an adenovirus, an adeno-related virus, a retrovirus, a herpes simplex virus and a vaccinia virus.

The vector includes a promoter operable in animal cells, for example, mammalian cells. Suitable promoters according to an embodiment include promoters derived from mammalian virus and promoters derived from the genome of mammalian cells, such as CMV (cytomegalovirus) promoter, U6 promoter and H1 promoter, MLV (Murine Leukemia Virus) LTR (Long terminal repeat) promoter, adenovirus early promoter, adenovirus late promoter, vaccinia virus 7.5K promoter, SV40 promoter, HSV tk promoter, RSV promoter, EF1 alpha promoter, metallothionein promoter, beta-actin promoter, promoter of human IL-2 gene, promoter of human IFN gene, promoter of human IL-4 gene, promoter of human lymphotoxin gene, promoter of human GM-CSF gene, human phosphoglycerate kinase (PGK) promoter, mouse phosphoglycerate kinase (PGK) promoter and sulvivin promoter.

In addition, in the vector, the aforementioned cytokine library sequence may be operably linked to a promoter. As used herein, the term “operably linked” refers to a functional linkage between a nucleic acid expression control sequence (e.g., a promoter, a signal sequence, or an array of transcriptional regulatory factor binding sites) and another nucleic acid sequence, and thereby the regulatory sequence controls the transcription and/or translation of the other nucleic acid sequence.

Another aspect provides a host cell comprising any one of the fusion polypeptides, polynucleotides, or vectors selected from the cytokine library.

The cytokines, transmembrane domains, vectors, and the like are as described in the aforementioned cytokine library.

The cells, e.g., eukaryotic cells, are cells of yeast, fungi, protozoa, plants, higher plants and insects, or amphibians, or cells of mammals such as CHO, HeLa, HEK293, and COS-1. For example, it may include cultured cells (in vitro), grafted cells and primary cell cultures (in vitro and ex vivo), and in vivo and a mammalian cell including a human, which are commonly used in the art. In addition, the organism may be yeast, fungi, protozoa, plants, higher plants and insects, amphibians, or mammals. In addition, the cells may be animal cells or plant cells.

Another aspect provides a cytokine library comprising the fusion polypeptide, the polynucleotide, the vector or the host cell.

As used herein, the term “cytokine library” refers to a collection containing cytokines having various biological activities, and individual members of the cytokine library may commonly include fusion polypeptides comprising cytokines, linkers, and transmembrane domains, polynucleotides, vectors or cells.

The cytokine library may be used for evaluating the functionality of cytokines against target cells, that is, screening for functional cytokines against target cells.

According to an embodiment, the cytokine library is introduced into various target cells including vascular endothelial cells and so on to provide continuous stimulation by cytokines to the target cells, so that changes of the biological activity including anti-angiogenesis, etc. could observed effectively and thus the functionality of cytokines on target cells could be screened.

Another aspect provides a method of screening cytokine comprising identifying any one change selected from the group consisting of a biological change of cell by a cytokine from the host cell, a change in the expression or activity of an exogenous or endogenous gene or protein and a combination thereof.

The cytokines, transmembrane domains, vectors, and the like are as described above.

According to an embodiment, it was possible to effectively induce a biological change in a host cell or a target cell by continuous stimulation of the cytokine by transfecting a vector comprising the cytokine library into a target cell and thus the functionality of cytokines could be effectively evaluated at a single cell level.

In one embodiment, the target cell is a cell isolated from a living body, and is not limited to its type, characteristics and origin, such as stem cells and somatic cells, and may generically refer to substantial all cells.

Transfection of the target cells is performed by introducing a vector containing a cytokine library of a conventional transfection method, for example, DEAE-dextran, calcium phosphate method, microinjection method, DNA-containing liposome method, lipopectamine-DNA complex method, etc. The transfection methods are known in the art.

In addition, in the screening method, the step of incubating the host cell with another biologically active substance may be further included. The biologically active substance may include, for example, a small molecule compound, an antibody, an antisense nucleotide, a short interfering RNA, a short hairpin RNA, a nucleic acid, a protein, a peptide, other extracts or natural products.

The step of confirming the biological change or the like may be performed by appropriately selecting a method known in the art according to the type of biological change to be screened. The biological change may be, for example, a series of reactions resulting from binding to a membrane receptor on the cell surface, and may be, for example, growth, differentiation, migration, cell senescence or apoptotic cell death.

### Advantageous Effects

According to the cytokine fusion polypeptide according to an aspect or the cytokine library containing the same, it enables continuous stimulation by cytokines to target cells to have an effect that can easily and accurately evaluate the function of cytokines at the level of a single cell.

### DESCRIPTION OF DRAWINGS

FIG. 1A shows the structure of a cytokine library according to an example and is a diagram schematically illustrating the structure of any one polypeptide or polynucleotide constituting the cytokine library.

FIG. 1B shows the structure of a cytokine library according to an example and is a diagram schematically illustrating a lentiviral vector prepared from any one polypeptide or polynucleotide constituting the cytokine library.

FIG. 2 is a diagram schematically illustrating a screening process for functional cytokines using a cytokine library according to an example.

FIG. 3 shows a result of screening cytokines affecting VEGF-dependent proliferation of HMVEC-L using the cytokine library according to an example.

FIG. 4 shows a result of confirming the effect of IL-5 on the proliferation of vascular endothelial cells induced by VEGF through MTT analysis; FIG. 4A shows a result of confirming the change in proliferative ability of vascular endothelial cells according to the concentration of IL-5; and FIG. 4B shows a result of confirming the change in proliferative capacity of vascular endothelial cells according to the treatment of IL-5 over time.

FIG. 5 shows a result of confirming the effect of IL-5 on the migration ability of vascular endothelial cells induced by VEGF through a wound-healing analysis; FIG. 5A shows a result of observing the closure of the wound through the Image J software module; and FIG. 5B shows a result of quantitatively analyzing the closure rate for the wound.

FIG. 6 shows a result of confirming the effect of IL-5 on the tube formation ability of vascular endothelial cells induced by VEGF through a tube formation assay; FIG. 6A shows a result of observing formed branch points and tubes by Image J analysis software; FIG. 6B shows a quantitative result of the total number of generated branch points; and FIG. 6C shows a result of confirming the effect of IL-5 on the tube formation ability of vascular endothelial cells induced by VEGF through tube formation assay and quantitatively showing the total length of the formed tube.

FIG. 7 shows a result of confirming the phosphorylation of STAT5 by IL-treatment through Western blotting.

FIG. 8A shows a result of confirming the effect of treatment of IL-5 on the anti-angiogenic effect induced by VEGF for HMVEC-L in which STAT5 was knocked down and checking the proliferation of vascular endothelial cells through MTT analysis.

FIG. 8B shows a result of confirming the effect of treatment of IL-5 on the anti-angiogenic effect induced by VEGF for HMVEC-L in which STAT5 was knocked down and checking the migration ability of vascular endothelial cells to wound-healing analysis.

FIG. 8C shows a result of confirming the effect of the treatment of IL-5 on the anti-angiogenesis effect induced by VEGF for HMVEC-L in which STAT5 was knocked down, and checking the tube formation ability of vascular endothelial cells through tube formation analysis.

### BEST MODE

Hereinafter, the present invention will be described in more detail through examples. However, these examples are for illustrative purposes only and the scope of the present invention is not limited to these examples.

### Example 1: Experimental Materials and Experimental Preparation

1-1. Construction of Cytokine Library

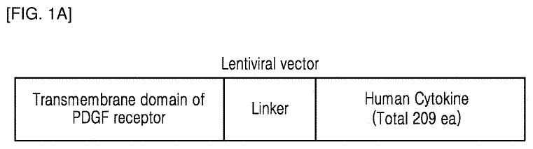

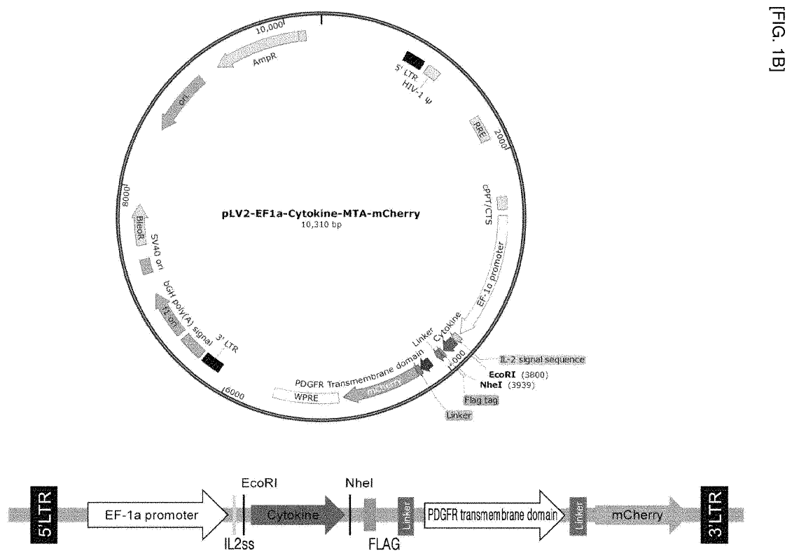

FIG. 1A and FIG. 1B show diagrams schematically illustrating the structure of any one polypeptide or polynucleotide constituting the cytokine library and a lentiviral vector prepared therefrom according to an example. Specifically, the human cytokine gene cDNA was obtained from the GE cytokine library. The PCR primers were designed to introduce each cDNA into a pair of Sfil restriction enzyme recognition sites compatible with the ends of each of the PCR amplification inserts in lentiviral vector pLV2-EF1a-MTA. After digesting the Sfil recognition region, the insert was individually ligated to a Sfil-cleaved lentiviral vector to construct a lentiviral cytokine plasmid for each cytokine. The insert was designed to include the structure of [cytokine-flexible linker-transmembrane domain] and the specific structure of the plasmid is shown in FIG. 1A. In addition, a plurality of cytokines applied in this Example are shown in Tables 1 to 7.

TABLE 1

| | | | | | Entrez | | NCBI. | NCBI. |
|---|---|---|---|---|---|---|---|---|
| | Antibiotic | Vector | Sequence | Host | Gene | Gene | GeneID | GeneSymbol |
| NO | Resistance | Name | Primer | Name | List | Symbol | List | List |
| 1 | Amp | pDrive | T7, sp6 | E. coli | 6355 | CCL8 [Homo | 6355 | CCL8 |
| | | | | | | sapiens] | | |
| 2 | Amp | pINCY | −21M13, M13 | E. coli | 9997 | SCO2 [Homo | 9997 | SCO2 |
| | | | reverse, T7, sp6 | | | sapiens] | | |
| 3 | Amp | pSPORT1 | T7, sp6, −21M13, | E. coli | 2323 | FLT3LG[Homo | 2323 | FLT3LG |
| | | | M13 reverse | | | sapiens] | | |
| 4 | Amp | pINCY | −21M13, M13 | E. coli | 1440 | CSF3[Homo | 1440 | CSF3 |
| | | | reverse, T7, sp6 | | | sapiens] | | |
| 5 | Amp | pCMV-SPORT6 | sp6, T7, −21M13, | DH10B TonA | 6357 | CCL13 [Homo | 6357 | CCL13 |
| | | | M13 reverse | | | sapiens] | | |
| 6 | Amp | pCMV-SPORT6 | sp6, T7, −21M13, | DH10B TonA | 131177 | FAM3D [Homo | 131177 | FAM3D |
| | | | M13 reverse | | | sapiens] | | |
| 7 | Amp | pCMV-SPORT6 | sp6, T7, −21M13, | DH10B TonA | 652 | BMP4 [Homo | 652 | BMP4 |
| | | | M13 reverse | | | sapiens] | | |
| 8 | Amp | pBluescriptR | T3, T7, −21M13, | DH10B | 51752 | ERAP1 [Homo | 51752 | ERAP1 |
| | | | M13 reverse | | | sapiens] | | |
| 9 | Amp | pCMV-SPORT6.ccdb | sp6, T7, −21M13, | DH10B TonA | 7424 | VEGFC [Homo | 7424 | VEGFC |
| | | | M13 reverse | | | apiens] | | |
| 10 | Amp | pCMV-SPORT6 | sp6, T7, −21M13, | DH10B | 6367 | CCL22 [Homo | 6367 | CCL22 |
| | | | M13 reverse | | | sapiens] | | |
| 11 | Amp | pCMV-SPORT6 | sp6, T7, −21M13, | DH10B | 2662 | GDF10 [Homo | 2662 | GDF10 |
| | | | M13 reverse | | | sapiens] | | |
| 12 | Amp | pDrive | T7, sp6 | E. coli | 9235 | IL32 [Homo | 9235 | IL32 |
| | | | | | | sapiens] | | |
| 13 | Amp | pINCY | −21M13, M13 | E. coli | 2257 | FGF12 [Homo | 2257 | FGF12 |
| | | | reverse, T7, sp6 | | | sapiens] | | |
| 14 | Amp | pDrive | T7, sp6 | E. coli | 54097 | FAM3B [Homo | 54097 | FAM3B |
| | | | | | | sapiens] | | |
| 15 | Amp | pBluescript | M13(−21), | E. coli | 3557 | IL1RN [Homo | 3557 | IL1RN |
| | | | M13 reverse, | | | sapiens] | | |
| | | | T7, T3 | | | | | |
| 16 | Amp | pCMV-SPORT6 | sp6, T7, −21M13, | DH10B TonA | 655 | BMP7 [Homo | 655 | BMP7 |
| | | | M13 reverse | | | sapiens] | | |
| 17 | Amp | pCMV-SPORT6 | sp6, T7, −21M13, | DH10B TonA | 3569 | IL6 [Homo | 3569 | IL6 |
| | | | M13 reverse | | | sapiens] | | |
| 18 | Amp | pCMV-SPORT6 | sp6, T7, −21M13, | DH10B TonA | 23529 | CLCF1 [Homo | 23529 | CLCF1 |
| | | | M13 reverse | | | sapiens] | | |
| 19 | Amp | pBluescriptR | T3, T7, −21M13, | DH10B | 3625 | INHBB [Homo | 3625 | INHBB |
| | | | M13 reverse | | | sapiens] | | |
| 20 | Amp | pCMV-SPORT6 | sp6, T7, −21M13, | DH10B | 26525 | IL1F5 [Homo | 26525 | IL1F5 |
| | | | M13 reverse | | | sapiens] | | |
| 21 | Amp | pCMV-SPORT6 | sp6, T7, −21M13, | DH10B | 6366 | CCL21 [Homo | 6366 | CCL21 |
| | | | M13 reverse | | | sapiens] | | |
| 22 | Amp | pCMV-SPORT6 | sp6, T7, −21M13, | DH10B | 8200 | GDF5 [Homo | 8200 | GDF5 |
| | | | M13 reverse | | | sapiens] | | |
| 23 | Amp | pINCY | −21M13, M13 | E. coli | 4982 | TNFRSF11B | 4982 | TNFRSF11B |
| | | | reverse, T7, sp6 | | | [Homo sapiens] | | |
| 24 | Amp | pINCY | −21M13, M13 | E. coli | 5154 | PDGFA [Homo | 5154 | PDGFA |
| | | | reverse, T7, sp6 | | | sapiens] | | |
| 25 | Amp | pINCY | −21M13, M13 | E. coli | 9560 | CCL4L1 [Homo | 9560, 388372 | CCL4L1, |
| | | | reverse, T7, sp6 | | | sapiens] | | CCL4L2 |
| 26 | Amp | pCMV-SPORT6 | sp6, T7, −21M13, | DH10B TonA | 6352 | CCL5 [Homo | 6352 | CCL5 |
| | | | M13 reverse | | | sapiens] | | |
| 27 | Amp | pCMV-SPORT6 | sp6, T7, −21M13, | DH10B TonA | 3576 | IL8 [Homo | 3576 | IL8 |
| | | | M13 reverse | | | sapiens] | | |
| 28 | Amp | pCMV-SPORT6 | sp6, T7, −21M13, | DH10B TonA | 6372 | CXCL6 [Homo | 6372 | CXCL6 |
| | | | M13 reverse | | | sapiens] | | |
| 29 | Amp | pBluescriptR | T3, T7, −21M13, | DH10B | 53342 | IL17D [Homo | 53342 | IL17D |
| | | | M13 reverse | | | sapiens] | | |
| 30 | Amp | pCMV-SPORT6 | sp6, T7, −21M13, | DH10B | 7205 | TRIP6 [Homo | 7205 | TRIP6 |
| | | | M13 reverse | | | sapiens] | | |
| 31 | Amp | pCMV-SPORT6 | sp6, T7, −21M13, | DH10B | 6363 | CCL19 [Homo | 6363 | CCL19 |
| | | | M13 reverse | | | sapiens] | | |
| 32 | Amp | pCMV-SPORT6 | sp6, T7, −21M13, | DH10B TonA | 8743 | TNFSF10 [Homo | 8743 | TNFSF10 |
| | | | M13 reverse | | | sapiens] | | |
| 33 | Amp | pBluescriptR | T3, T7, −21M13, | DH10B | 90865 | IL33 [Homo | 90865 | IL33 |
| | | | M13 reverse | | | sapiens] | | |
| 34 | Amp | pDrive | T7, sp6 | E. coli | 6368 | CCL23 [Homo | 6368 | CCL23 |
| | | | | | | sapiens] | | |
| 35 | Amp | pINCY | −21M13, M13 | E. coli | 7857 | SCG2 [Homo | 7857 | SCG2 |
| | | | reverse, T7, sp6 | | | sapiens] | | |

TABLE 2

| | | | | | Entrez | | NCBI. | NCBI. |
|---|---|---|---|---|---|---|---|---|
| | Antibiotic | Vector | Sequence | Host | Gene | Gene | GeneID | GeneSymbol |
| NO | Resistance | Name | Primer | Name | List | Symbol | List | List |
| 36 | Amp | pINCY | −21M13, M13 | E. coli | 970 | CD70 [Homo sapiens] | 970 | CD70 |
| | | | reverse, T7, sp6 | | | | | |
| 37 | Amp | pINCY | −21M13, M13 | E. coli | 51192 | CKLF [Homo sapiens] | 51192 | CKLF |
| | | | reverse, T7, sp6 | | | | | |
| 38 | Amp | pCMV-SPORT6 | sp6, T7, −21M13, | DH10B | 11009 | IL24 [Homo sapiens] | 11009 | IL24 |
| | | | M13 reverse | TonA | | | | |
| 39 | Amp | pCMV-SPORT6 | sp6, T7, −21M13, | DH10B | 1890 | TYMP [Homo sapiens] | 1890 | TYMP |
| | | | M13 reverse | TonA | | | | |
| 40 | Amp | pCMV-SPORT6 | sp6, T7, −21M13, | DH10B | 2896 | GRN [Homo sapiens] | 2896 | GRN |
| | | | M13 reverse | | | | | |
| 41 | Amp | pCMV-SPORT6 | sp6, T7, −21M13, | DH10B | 653 | BMP5 [Homo sapiens] | 653 | BMP5 |
| | | | M13 reverse | | | | | |
| 42 | Amp | pCMV-SPORT6 | sp6, T7, −21M13, | DH10B | 3603 | IL16 [Homo sapiens] | 3603 | IL16 |
| | | | M13 reverse | | | | | |
| 43 | Amp | pCMV-SPORT6 | sp6, T7, −21M13, | DH10B | 7044 | LEFTY2 [Homo | 7044 | LEFTY2 |
| | | | M13 reverse | | | sapiens] | | |
| 44 | Amp | pBluescriptR | T3, T7, −21M13, | DH10B | 1442, 1443, | CSH1 [Homo sapiens], | 1442 | CSH1 |
| | | | M13 reverse | TonA | 81848 | CSH2 [Homo sapiens], | | |
| | | | | | | SPRY4 [Homo sapiens] | | |
| 45 | Amp | pINCY | −21M13, M13 | E. coli | 10572 | SIVA1 [Homo sapiens] | 10572 | SIVA1 |
| | | | reverse, T7, sp6 | | | | | |
| 46 | Amp | pINCY | −21M13, M13 | E. coli | 3589 | IL11 [Homo sapiens] | 3589 | IL11 |
| | | | reverse, T7, sp6 | | | | | |
| 47 | Amp | pBSK-.2 | T7, T3 | E. coli | 6386 | SDCBP [Homo sapiens] | 6386 | SDCBP |
| 48 | Amp | pCMV-SPORT6 | sp6, T7, −21M13, | DH10B | 3552 | IL1A [Homo sapiens] | 3552 | IL1A |
| | | | M13 reverse | TonA | | | | |
| 49 | Amp | pCMV-SPORT6 | sp6, T7, −21M13, | DH10B | 10148 | EBI3 [Homo sapiens] | 10148 | EBI3 |
| | | | M13 reverse | TonA | | | | |
| 50 | Amp | pCMV-SPORT6 | sp6, T7, −21M13, | DH10B | 3600 | LL15 [Homo sapiens] | 3600 | IL15 |
| | | | M13 reverse | TonA | | | | |
| 51 | Amp | pCMV-SPORT6 | sp6, T7, −21M13, | DH10B | 2277 | FIGF [Homo sapiens] | 2277 | FIGF |
| | | | M13 reverse | | | | | |
| 52 | Amp | pCMV-SPORT6 | sp6, T7, −21M13, | DH10B | 92304 | SCGB3A1 [Homo | 92304 | SCGB3A1 |
| | | | M13 reverse | | | sapiens] | | |
| 53 | Amp | pCMV-SPORT6 | sp6, T7, −21M13, | DH10B | 6387 | CXCL12 [Homo | 6387 | CXCL12 |
| | | | M13 reverse | | | sapiens] | | |
| 54 | Amp | pINCY | −21M13, M13 | E. coli | 6364 | CCL20 [Homo sapiens] | 6364 | CCL20 |
| | | | reverse, T7, sp6 | | | | | |
| 55 | Amp | pINCY | −21M13, M13 | E. coli | 4049 | LTA [Homo sapiens] | 4049 | LTA |
| | | | reverse, T7, sp6 | | | | | |
| 56 | Amp | pSPORT1 | T7, sp6, −21M13, | E. coli | 64388 | GREM2 [Homo | 64388 | GREM2 |
| | | | M13 reverse | | | sapiens] | | |
| 57 | Amp | pINCY | −21M13, M13 | E. coli | 5197 | PF4V1 [Homo sapiens] | 5197 | PF4V1 |
| | | | reverse, T7, sp6 | | | | | |
| 58 | Amp | pCMV-SPORT6 | sp6, T7, −21M13, | DH10B | 3553 | IL1B [Homo sapiens] | 3553 | IL1B |
| | | | M13 reverse | TonA | | | | |
| 59 | Amp | pCMV-SPORT6 | sp6, T7, −21M13, | DH10B | 116173 | CMTM5 [Homo | 116173 | CMTM5 |
| | | | M13 reverse | TonA | | sapiens] | | |
| 60 | Amp | pCMV-SPORT6 | sp6, T7, −21M13, | DH10B | 6351 | CCL4 [Homo sapiens] | 6351 | CCL4 |
| | | | M13 reverse | | | | | |
| 61 | Amp | pCMV-SPORT6 | sp6, T7, −21M13, | DH10B | 5155 | PDGFB [Homo sapiens] | 5155 | PDGFB |
| | | | M13 reverse | | | | | |
| 62 | Amp | pBluescriptR | T3, T7, −21M13, | DH10B | 58191 | CXCL16 [Homo | 58191 | CXCL16 |
| | | | M13 reverse | | | sapiens] | | |
| 63 | Amp | pBluescriptR | T3, T7, −21M13, | DH10B | 113540 | CMTM1 [Homo | 113540 | CMTM1 |
| | | | M13 reverse | | | sapiens] | | |
| 64 | Amp | pINCY | −21M13, M13 | E. coli | 3084 | NRG1 [Homo sapiens] | 3084 | NRG1 |
| | | | reverse, T7, sp6 | | | | | |
| 65 | Amp | pINCY | −21M13, M13 | E. coli | 29949 | IL19 [Homo sapiens] | 29949 | IL19 |
| | | | reverse, T7, sp6 | | | | | |
| 66 | Amp | pINCY | −21M13, M13 | E. coli | 650 | BMP2 [Homo sapiens] | 650 | BMP2 |
| | | | reverse, T7, sp6 | | | | | |
| 67 | Amp | pCMV-SPORT6 | sp6, T7, −21M13, | DH10B | 6347 | CCL2 [Homo sapiens] | 6347 | CCL2 |
| | | | M13 reverse | TonA | | | | |
| 68 | Amp | pCMV-SPORT6 | sp6, T7, −21M13, | DH10B | 6398 | SECTM1 [Homo | 6398 | SECTM1 |
| | | | M13 reverse | TonA | | sapiens] | | |
| 69 | Amp | pBluescriptR | T3, T7, −21M13, | DH10B | 8740 | TNFSF14 [Homo | 8740 | TNFSF14 |
| | | | M13 reverse | | | sapiens] | | |
| 70 | Amp | pCMV-SPORT6 | sp6, T7, −21M13, | DH10B | 10637 | LEFTY1 [Homo | 10637 | LEFTY1 |
| | | | M13 reverse | | | sapiens] | | |

TABLE 3

| | | | | | Entrez | | NCBI. | NCBI. |
|---|---|---|---|---|---|---|---|---|
| | Antibiotic | Vector | Sequence | Host | Gene | Gene | GeneID | GeneSymbol |
| NO | Resistance | Name | Primer | Name | List | Symbol | List | List |
| 71 | Amp | pCMV-SPORT6 | sp6, T7, −21M13, | DH10B | 54918 | CMTM6 [Homo | 54918 | CMTM6 |
| | | | M13 reverse | TonA | | sapiens] | | |
| 72 | Amp | pBluescriptR | T3, T7, −21M13, | DH10B | 123920 | CMTM3 [Homo | 123920 | CMTM3 |
| | | | M13 reverse | | | sapiens] | | |
| 73 | Amp | pINCY | −21M13, M13 reverse, | E. coli | 5827 | PXMP2 [Homo sapiens] | 5827 | PXMP2 |
| | | | T7, sp6 | | | | | |
| 74 | Amp | pSPORT1 | T7, sp6, −21M13, | E. coli | 8741 | TNFSF13 [Homo | 8741 | TNFSF13 |
| | | | M13 reverse | | | sapiens] | | |
| 75 | Amp | pINCY | −21M13, M13 reverse, | E. coli | 152189 | CMTM8 [Homo | 152189 | CMTM8 |
| | | | T7, sp6 | | | sapiens] | | |
| 76 | Amp | pCMV-SPORT6 | sp6, T7, −21M13, | DH10B | 9577 | BRE [Homo sapiens] | 9577 | BRE |
| | | | M13 reverse | | | | | |
| 77 | Amp | pCMV-SPORT6 | sp6, T7, −21M13, | DH10B | 2919 | CXCL1 [Homo sapiens] | 2919 | CXCL1 |
| | | | M13 reverse | TonA | | | | |
| 78 | Amp | pCMV-SPORT6 | sp6, T7, −21M13, | DH10B | 8565 | YARS [Homo sapiens] | 8565 | YARS |
| | | | M13 reverse | TonA | | | | |
| 79 | Amp | pCMV-SPORT6 | sp6, T7, −21M13, | DH10B | 113540 | CMTM1 [Homo | 23433 | RHOQ |
| | | | M13 reverse | TonA | | sapiens] | | |
| 80 | Amp | pCMV-SPORT6 | sp6, T7, −21M13, | DH10B | 414062 | CCL3L3 [Homo | 6349 | CCL3L1 |
| | | | M13 reverse | | | sapiens] | | |
| 81 | Amp | pCMV-SPORT6 | sp6, T7, −21M13, | DH10B | 5473 | PPBP [Homo sapiens] | 5473 | PPBP |
| | | | M13 reverse | | | | | |
| 82 | Amp | pBluescriptR | T3, T7, −21M13, | DH10B | 85480 | TSLP [Homo sapiens] | 85480 | TSLP |
| | | | M13 reverse | | | | | |
| 83 | Amp | pCMV-SPORT6 | sp6, T7, −21M13, | DH10B | 10447 | FAM3C [Homo sapiens] | 10447 | FAM3C |
| | | | M13 reverse | TonA | | | | |
| 84 | Amp | pCMV-SPORT6 | sp6, T7, −21M13, | DH10B | 1489 | CTF1 [Homo sapiens] | 1489 | CTF1 |
| | | | M13 reverse | | | | | |
| 85 | Amp | pDNR-Dual | M13(−21), T7 | DH10B | 3567 | IL5 [Homo sapiens] | 3567 | IL5 |
| | | | | TonA | | | | |
| 86 | Amp | pDNR-Dual | M13(−21), T7 | DH10B | 3558 | IL2 [Homo sapiens] | 3558 | IL2 |
| | | | | TonA | | | | |
| 87 | Amp | pCMV-SPORT6.1 | M13(−21), M13 reverse, sp6 | DH10B | 2688 | GH1 [Homo sapiens] | 2688 | GH1 |
| | | | | TonA | | | | |
| 88 | Amp | pCR4-TOPO | M13 (−21), M13 reverse, | DH10B | 3441 | IFNA4 [Homo sapiens] | 3441 | IFNA4 |
| | | | T7, 13 | TonA | | | | |
| 89 | Amp | pCR4-TOPO | M13 (−21), M13 reverse, | DH10B | 146223 | CMTM4 [Homo | 146223 | CMTM4 |
| | | | T7, 13 | TonA | | sapiens] | | |
| 90 | Amp | pCMV-SPORT6 | sp6, T7, −21M13, | DH10B | 7422 | VEGFA [Homo sapiens] | 7422 | VEGFA |
| | | | M13 reverse | TonA | | | | |
| 91 | Amp | pDNR-Dual | M13(−21), T7 | DH10B | 3605 | IL17A [Homo sapiens] | 3605 | IL17A |
| | | | | TonA | | | | |
| 92 | Amp | pDNR-Dual | M13(−21), T7 | DH10B | 59067 | IL21 [Homo sapiens] | 59067 | IL21 |
| | | | | TonA | | | | |
| 93 | Amp | pBluescriptR | T3, T7, −21M13, | DH10B | 6696 | SPP1 [Homo sapiens] | 6696 | SPP1 |
| | | | M13 reverse | | | | | |
| 94 | Amp | pCMV-SPORT6 | sp6, T7, −21M13, | DH10B | 10715 | LASS1 [Homo sapiens] | 10715 | LASS1 |
| | | | M13 reverse | | | | | |
| 95 | Amp | pCR4-TOPO | M13 (−21), Ml3 reverse, | DH10B | 3439 | IFNA1 [Homo sapiens] | 3439 | IFNA1 |
| | | | T7, T3 | TonA | | | | |
| 96 | Amp | pCR4-TOPO | M13 (−21), M13 reverse, | DH10B | 282618 | IL29 [Homo sapiens] | 282618 | IL29 |
| | | | T7, T3 | TonA | | | | |
| 97 | Amp | pCR4-TOPO | M13 (−21), M13 reverse, | DH10B | 3442 | IFNA5 [Homo sapiens] | 3442 | IFNA5 |
| | | | T7,T3 | TonA | | | | |
| 98 | Amp | pCMV-SPORT6 | sp6, T7, −21M13, M13 | DH10B | 6358 | CCL14 [Homo sapiens] | 6358 | CCL14 |
| | | | reverse | | | | | |
| 99 | Amp | pBluescriptR | T3, T7, −21M13, | DH10B | 5295 | PIK3R1 [Homo sapiens] | 5295 | PIK3R1 |
| | | | M13 reverse | TonA | | | | |
| 100 | Amp | pCMV-SPORT6 | sp6, T7, −21M13, | DH10B | 6373 | CXCL11 [Homo | 6373 | CXCL11 |
| | | | M13 reverse | | | sapiens] | | |
| 101 | Amp | pCR4-TOPO | M13 (−21), M13 reverse, | DH10B | 2660 | MSTN [Homo sapiens] | 2660 | MSTN |
| | | | T7, T3 | TonA | | | | |
| 102 | Amp | pCR4-TOPO | M13 (−21), M13 reverse, | DH10B | 3444 | IFNA7 [Homo sapiens] | 3444 | IFNA7 |
| | | | T7, T3 | TonA | | | | |
| 103 | Amp | pCMV-SPORT6 | sp6, T7, −21M13, | DH10B | 3574 | IL7 [Homo sapiens] | 3574 | IL7 |
| | | | M13 reverse | | | | | |
| 104 | Amp | pDNR-Dual | M13(−21), T7 | DH10B | 50616 | IL22 [Homo sapiens] | 50616 | IL22 |
| | | | | TonA | | | | |
| 105 | Amp | pCMV-SPORT6 | sp6, T7, −21M13, | DH10B | 10135 | NAMPT [Homo sapiens] | 10135 | NAMPT |
| | | | M13 reverse | TonA | | | | |

TABLE 4

| | | | | | Entrez | | NCBI. | NCBI. |
|---|---|---|---|---|---|---|---|---|
| | Antibiotic | Vector | Sequence | Host | Gene | Gene | GeneID | GeneSymbol |
| NO | Resistance | Name | Primer | Name | List | Symbol | List | List |
| 106 | Amp | pCR4-TOPO | M13 (−21), M13 reverse, | DH10B | 3440 | IFNA2 [Homo sapiens] | 3440 | IFNA2 |
| | | | T7, T3 | TonA | | | | |
| 107 | Amp | pCR4-TOPO | M13 (−21), M13 reverse, | DH10B | 3448 | IFNA14 [Homo | 3448 | IFNA14 |
| | | | T7, T3 | TonA | | sapiens] | | |
| 108 | Amp | pCR4-TOPO | M13 (−21), M13 reverse, | DH10B | 3976 | LIF [Homo sapiens] | 3976 | LIF |
| | | | T7, T3 | TonA | | | | |
| 109 | Amp | pDNR-Dual | M13(−21), T7 | DH10B | 51561 | IL23A [Homo sapiens] | 51561 | IL23A |
| | | | | TonA | | | | |
| 110 | Amp | pDNR-Dual | M13(−21), T7 | DH10B | 3593 | IL12B [Homo sapiens] | 3593 | IL12B |
| | | | | TonA | | | | |
| 111 | Amp | pBluescriptR | T3, T7, −21M13, | DH10B | 4283 | CXCL9 [Homo | 4283 | CXCL9 |
| | | | M13 reverse | TonA | | sapiens] | | |
| 112 | Amp | pCMV-SPORT6 | sp6, T7, −21M13, | DH10B | 1437 | CSF2 [Homo sapiens] | 1437 | CSF2 |
| | | | M13 reverse | TonA | | | | |
| 113 | Amp | pCR4-TOPO | M13 (−21), M13 reverse, | DH10B | 9966 | TNFSF15 [Homo | 9966 | TNFSF15 |
| | | | T7, T3 | TonA | | sapiens] | | |
| 114 | Amp | pCR4-TOPO | M13 (−21), M13 reverse, | DH10B | 2658 | GDF2 [Homo sapiens] | 2658 | GDF2 |
| | | | T7, T3 | TonA | | | | |
| 115 | Amp | pBluescriptR | T3, T7, −21M13, | DH10B | 6579 | SLCO1A2 [Homo | 6579 | SLCO1A2 |
| | | | M13 reverse | | | sapiens] | | |
| 116 | Amp | pCMV-SPORT6 | sp6, T7, −21M13, | DH10B | 656 | BMP8B [Homo | 656 | BMP8B |
| | | | M13 reverse | TonA | | sapiens] | | |
| 117 | Amp | pCR4-TOPO | M13 (−21), M13 reverse, | DH10B | 8600 | TNFSF11 [Homo | 8600 | TNFSF11 |
| | | | T7, T3 | TonA | | sapiens] | | |
| 118 | Amp | pCR4-TOPO | M13 (−21), M13 reverse, | DH10B | 2056 | EPO [Homo sapiens] | 2056 | EPO |
| | | | T7, T3 | TonA | | | | |
| 119 | Amp | pCR4-TOPO | M13 (−21), M13 reverse, | DH10B | 10344 | CCL26 [Homo sapiens] | 10344 | CCL26 |
| | | | T7, T3 | TonA | | | | |
| 120 | Amp | pCMV-SPORT6 | sp6, T7, −21M13, | DH10B | 4282 | MIF [Homo sapiens] | 4282 | MIF |
| | | | M13 reverse | TonA | | | | |
| 121 | Amp | pDNR-Dual | M13(−21), T7 | DH10B | 3565 | IL4 [Homo sapiens] | 3565 | IL4 |
| | | | | TonA | | | | |
| 122 | Amp | pCR4-TOPO | M13 (−21), M13 reverse, | DH10B | 4254 | KITLG [Homo sapiens] | 4254 | KITLG |
| | | | T7, T3 | TonA | | | | |
| 123 | Amp | pCR4-TOPO | M13 (−21), M13 reverse, | DH10B | 50604 | IL20 [Homo sapiens] | 50604 | IL20 |
| | | | T7, T3 | TonA | | | | |
| 124 | Amp | pCR4-TOPO | M13 (−21), M13 reverse, | DH10B | 944 | TNFSF8 [Homo | 944 | TNFSF8 |
| | | | T7, T3 | TonA | | sapiens] | | |
| 125 | Amp | pCR4-TOPO | M13 (−21), M13 reverse, | DH10B | 26585 | GREM1 [Homo | 26585 | GREM1 |
| | | | T7, T3 | TonA | | sapiens] | | |
| 126 | Amp | pDNR-Dual | M13(−21), T7 | DH10B | 3578 | IL9 [Homo sapiens] | 3578 | IL9 |
| | | | | TonA | | | | |
| 127 | Amp | pDNR-Dual | M13(−21), T7 | DH10B | 3562 | IL3 [Homo sapiens] | 3562 | IL3 |
| | | | | TonA | | | | |
| 128 | Amp | pCMV-SPORT6.1 | M13(−21), M13 reverse, | DH10B | 1270 | CNTF [Homo sapiens] | 1270 | CNTF |
| | | | sp6 | TonA | | | | |
| 129 | Amp | pCR4-TOPO | M13 (−21), M13 reverse, | DH10B | 959 | CD40LG [Homo | 959 | CD40LG |
| | | | T7, T3 | TonA | | sapiens] | | |
| 130 | Amp | pCR4-TOPO | M13 (−21), M13 reverse, | DH10B | 5196 | PF4 [Homo sapiens] | 5196 | PF4 |
| | | | T7, T3 | TonA | | | | |
| 131 | Amp | pCR4-TOPO | M13 (−21), M13 reverse, | DH10B | 27177 | IL1F8 [Homo sapiens] | 27177 | IL1F8 |
| | | | T7, T3 | TonA | | | | |
| 132 | Amp | pCR4-TOPO | M13 (−21), M13 reverse, | DH10B | 3592 | IL12A [Homo sapiens] | 3592 | IL12A |
| | | | T7, T3 | TonA | | | | |
| 133 | Amp | pCR4-TOPO | M13 (−21), M13 reverse, | DH10B | 6346 | CCL1 [Homo sapiens] | 6346 | CCL1 |
| | | | T7, T3 | TonA | | | | |
| 134 | Amp | pPCR-Script Amp | M13(−21), M13 reverse, | XL10 Gold | 3443 | IFNA6 [Homo sapiens] | 3443 | IFNA6 |
| | | SK(+) | T7, T3 | | | | | |
| 135 | Amp | pCR4-TOPO | M13 (−21), M13 reverse, | DH10B | 6354 | CCL7 [Homo sapiens] | 6354 | CCL7 |
| | | | T7, T3 | TonA | | | | |
| 136 | Amp | pCR4-TOPO | M13 (−21), M13 reverse, | DH10B | 9210 | BMP15 [Homo | 9210 | BMP15 |
| | | | T7, T3 | TonA | | sapiens] | | |
| 137 | Amp | pCR4-TOPO | M13 (−21), M13 reverse, | DH10B | 3447 | IFNA13 [Homo | 3447 | IFNA13 |
| | | | T7, T3 | TonA | | sapiens] | | |
| 138 | Amp | pCR4-TOPO | M13 (−21), M13 reverse, | DH10B | 64806 | IL25 [Homo sapiens] | 64806 | IL25 |
| | | | T7, T3 | TonA | | | | |
| 139 | Amp | pPCR-Script Amp | M13(−21), M13 reverse, | XL10 Gold | 56477 | CCL28 [Homo sapiens] | 56477 | CCL28 |
| | | SK(+) | T7, T3 | | | | | |
| 140 | Amp | pCR4-TOPO | M13 (−21), M13 reverse, | DH10B | 7066 | THPO [Homo sapiens] | 7066 | THPO |
| | | | T7, T3 | TonA | | | | |

TABLE 5

| | | | | | Entrez | | NCBI. | NCBI. |
|---|---|---|---|---|---|---|---|---|
| | Antibiotic | Vector | Sequence | Host | Gene | Gene | GeneID | GeneSymbol |
| NO | Resistance | Name | Primer | Name | List | Symbol | List | List |
| 141 | Amp | pCR4-TOPO | M13 (−21), M13 reverse, | DH10B TonA | 8995 | TNFSF18 [Homo | 8995 | TNFSF18 |
| | | | T7, T3 | | | sapiens] | | |
| 142 | Amp | pCR4-TOPO | M13 (−21), M13 reverse, | DH10B TonA | 27189 | IL17C [Homo | 27189 | IL17C |
| | | | T7, T3 | | | sapiens] | | |
| 143 | Amp | pCR4-TOPO | M13 (−21), M13 reverse, | DH10B TonA | 57152 | SLURP1 [Homo | 57152 | SLURP1 |
| | | | T7, T3 | | | sapiens] | | |
| 144 | Amp | pCR4-TOPO | M13 (−21), M13 reverse, | DH10B TonA | 282616 | IL28A [Homo | 282616 | IL28A |
| | | | T7, T3 | | | sapiens] | | |
| 145 | Amp | pCR4-TOPO | M13 (−21), M13 reverse, | DH10B TonA | 1896 | EDA [Homo sapiens] | 1896 | EDA |
| | | | T7, T3 | | | | | |
| 146 | Amp | pCR4-TOPO | M13 (−21), M13 reverse, | DH10B TonA | 3452 | IFNA21 [Homo | 3452 | IFNA21 |
| | | | T7, T3 | | | sapiens] | | |
| 147 | Amp | pCR4-TOPO | M13 (−21), M13 reverse, | DH10B TonA | 8744 | TNFSF9 [Homo | 8744 | TNFSF9 |
| | | | T7, T3 | | | sapiens] | | |
| 148 | Amp | pPCR-Script Amp | M13(−21), M13 reverse, | DH10B TonA | 4050 | LTB [Homo sapiens] | 4050 | LTB |
| | | SK(+) | T7, T3 | | | | | |
| 149 | Amp | pCR4-TOPO | M13 (−21), M13 reverse, | DH10B TonA | 3467 | IFNW1 [Homo | 3467 | IFNW1 |
| | | | T7, T3 | | | sapiens] | | |
| 150 | Amp | pCR4-TOPO | M13 (−21), M13 reverse, | DH10B TonA | 6370 | CCL25 [Homo | 6370 | CCL25 |
| | | | T7, T3 | | | sapiens] | | |
| 151 | Amp | pCR4-TOPO | M13 (−21), M13 reverse, | DH10B TonA | 3445 | IFNA8 [Homo | 3445 | IFNA8 |
| | | | T7, T3 | | | sapiens] | | |
| 152 | Amp | pPCR-Script Amp | M13(−21), M13 reverse, | DH10B TonA | 9350 | CER1 [Homo sapiens | 9350 | CER1 |
| | | SK(+) | T7, T3 | | | | | |
| 153 | Amp | pCR4-TOPO | M13 (−21), M13 reverse, | DH10B TonA | 353500 | BMP8A [Homo | 353500 | BMP8A |
| | | | T7, T3 | | | sapiens] | | |
| 154 | Amp | pCR4-TOPO | M13 (−21), M13 reverse, | DH10B TonA | 27302 | BMP10 [Homo | 27302 | BMP10 |
| | | | T7, T3 | | | sapiens] | | |
| 155 | Amp | pCR4-TOPO | M13 (−21), M13 reverse, | DH10B TonA | 4838 | NODAL [Homo | 4838 | NODAL |
| | | | T7, T3 | | | sapiens] | | |
| 156 | Amp | pCR4-TOPO | M13 (−21), M13 reverse, | DH10B TonA | 6361 | CCL17 [Homo | 6361 | CCL17 |
| | | | T7, T3 | | | sapiens] | | |
| 157 | Amp | pPCR-Script Amp | M13(−21), M13 reverse, | XL10 Gold | 3446 | IFNA10 [Homo | 3446 | IFNA10 |
| | | SK(+) | T7, T3 | | | sapiens] | | |
| 158 | Amp | pPCR-Script Amp | M13(−21), M13 reverse, | DH10B TonA | 56603 | CYP26B1 [Homo | 56603 | CYP26B1 |
| | | SK(+) | T7, T3 | | | sapiens] | | |
| 159 | Amp | pCR4-TOPO | M13 (−21), M13 reverse, | DH10B TonA | 200734 | SPRED2 [Homo | 200734 | SPRED2 |
| | | | T7, T3 | | | sapiens] | | |
| 160 | Amp | pCR4-TOPO | M13 (−21), M13 reverse, | DH10B TonA | 51384 | WNT16 [Homo | 51384 | WNT16 |
| | | | T7, T3 | | | sapiens] | | |
| 161 | Amp | pCR4-TOPO | M13 (−21), M13 reverse, | DH10B TonA | 2255 | FGF10 [Homo | 2255 | FGF10 |
| | | | T7, T3 | | | sapiens] | | |
| 162 | Amp | pPCR-Script Amp | M13(−21), M13 reverse, | DH10B TonA | 6362 | CCL18 [Homo | 6362 | CCL18 |
| | | SK(+) | T7,T3 | | | sapiens] | | |
| 163 | Amp | pCR4-TOPO | M13 (−21), M13 reverse, | DH10B TonA | 282617 | IL28B [Homo | 282617 | IL28B |
| | | | T7, T3 | | | sapiens] | | |
| 164 | Amp | pCMV-SPORT6 | sp6, T7, −21M13, | DH10B | 11146 | GLMN [Homo | 11146 | GLMN |
| | | | M13 reverse | | | sapiens] | | |
| 165 | Cam | pOTB7 | −21M13, M13 reverse, | GeneHogs | 7423 | VEGFB [Homo | 7423 | VEGFB |
| | | | sp6, T7 | DH10B | | sapiens] | | |
| 166 | Cam | pDNR-LIB | −21M13 | GeneHogs | 3606 | IL18 [Homo sapiens] | 3606 | IL18 |
| | | | | DH10B | | | | |
| 167 | Cam | pDNR-LIB | −21M13 | DH10B TonA | 112744 | IL17F [Homo | 112744 | IL17F |
| | | | | | | sapiens] | | |
| 168 | Cam | pDNR-LIB | −21M13 | GeneHogs | 10563 | CXCL13 [Homo | 10563 | CXCL13 |
| | | | | DH10B | | sapiens] | | |
| 169 | Cam | pDNR-LIB | −21M13 | GeneHogs | 2921 | CXCL3 [Homo | 2921 | CXCL3 |
| | | | | DH10B | | sapiens] | | |
| 170 | Cam | pOTB7 | −21M13, M13 reverse, | GeneHogs | 146225 | CMTM2 [Homo | 146225 | CMTM2 |
| | | | sp6, T7 | DH10B | | sapiens] | | |
| 171 | Cam | pDNR-LIB | −21M13 | GeneHogs | 27178 | IL1F7 [Homo | 27178 | IL1F7 |
| | | | | DH10B | | sapiens] | | |
| 172 | Cam | pOTB7 | −21M13, M13 reverse, | GeneHogs | 9518 | GDF15 [Homo | 9518 | GDF15 |
| | | | sp6, T7 | DH10B | | sapiens] | | |
| 173 | Cam | pOTB7 | −21M13, M13 reverse, | GeneHogs | 9547 | CXCL14 [Homo | 9547 | CXCL14 |
| | | | sp6, T7 | DH10B | | sapiens] | | |
| 174 | Cam | pOTB7 | −21M13, M13 reverse, | GeneHogs | 5728 | PTEN [Homo | 5728 | PTEN |
| | | | sp6, T7 | DH10B | | sapiens] | | |
| 175 | Cam | pDNR-LIB | −21M13 | GeneHogs | 6374 | CXCL5 [Homo | 6374 | CXCL5 |
| | | | | DH10B | | sapiens] | | |

TABLE 6

| | | | | | Entrez | | NCBI. | NCBI. |
|---|---|---|---|---|---|---|---|---|
| | Antibiotic | Vector | Sequence | Host | Gene | Gene | GeneID | GeneSymbol |
| NO | Resistance | Name | Primer | Name | List | Symbol | List | List |
| 176 | Cam | pDNR- | −21M13 | GeneHogs | 3627 | CXCL10 [Homo sapiens] | 3627 | CXCL10 |
| | | LIB | | DH10B | | | | |
| 177 | Cam | pOTB7 | −21M13, M13 reverse, | GeneHogs | 356 | FASLG [Homo sapiens] | 356 | FASLG |
| | | | sp6, T7 | DH10B | | | | |
| 178 | Cam | pOTB7 | −21M13, M13 reverse, | GeneHogs | 6376 | CX3CL1 [Homo sapiens] | 6376 | CX3CL1 |
| | | | sp6, T7 | DH10B | | | | |
| 179 | Cam | pOTB7 | −21M13, M13 reverse, | GeneHogs | 8717, 8741 | TNFSF13 [Homo sapiens], | 8717 | TRADD |
| | | | sp6, T7 | DH10B | | TRADD [Homo sapiens] | | |
| ISO | Cam | pOTB7 | −21M13, M13 reverse, | GeneHogs | 3623 | INHA [Homo sapiens] | 3623 | INHA |
| | | | sp6, T7 | DH10B | | | | |
| 181 | Cam | pOTB7 | −21M13, M13 reverse, | GeneHogs | 4192 | MDK [Homo sapiens] | 4192 | MDK |
| | | | sp6, T7 | DH10B | | | | |
| 182 | Cam | pOTB7 | −21M13, M13 reverse, | GeneHogs | 2920 | CXCL2 [Homo sapiens] | 2920 | CXCL2 |
| | | | sp6, T7 | DH10B | | | | |
| 183 | Cam | pDNR- | −21M13 | GeneHogs | 5617 | PRL [Homo sapiens] | 5617 | PRL |
| | | LIB | | DH10B | | | | |
| 184 | Cam | pDNR- | −21M13 | GeneHogs | 246778 | IL27 [Homo sapiens] | 246778 | IL27 |
| | | LIB | | DH10B | | | | |
| 185 | Cam | pDNR- | −21M13 | DH10B TonA | 3458 | IFNG [Homo sapiens] | 3458 | IFNG |
| | | LIB | | | | | | |
| 186 | Cam | pOTB7 | −21M13, M13 reverse, | GeneHogs | 10131 | TRAP1 [Homo sapiens] | 10131 | TRAP1 |
| | | | sp6, T7 | DH10B | | | | |
| 187 | Cam | pOTB7 | −21M13, M13 reverse, | GeneHogs | 414062 | CCL3L3 [Homo sapiens], | 414062 | CCL3L3 |
| | | | sp6, T7 | DH10B | | CCL3L3 [Homo sapiens] | | |
| 188 | Cam | pDNR- | −21M13 | GeneHogs | 374 | AREG [Homo sapiens] | 374 | AREG |
| | | LIB | | DH10B | | | | |
| 189 | Cam | pOTB7 | −21M13, M13 reverse, | GeneHogs | 112616 | CMTM7 [Homo sapiens] | 112616 | CMTM7 |
| | | | sp6, T7 | DH10B | | | | |
| 190 | Cam | pDNR- | −21M13 | GeneHogs | 1443 | CSH2 [Homo sapiens] | 1443 | CSH2 |
| | | LIB | | DH10B | | | | |
| 191 | Cam | pDNR- | −21M13 | DH10B TonA | 6846 | XCL2 [Homo sapiens] | 6846 | XCL2 |
| | | LIB | | | | | | |
| 192 | Cam | pOTB7 | −21M13, M13 reverse, | GeneHogs | 2821 | GPI [Homo sapiens] | 2821 | GPI |
| | | | sp6, T7 | DH10B | | | | |
| 193 | Cam | pOTB7 | −21M13, M13 reverse, | GeneHogs | 7356 | SCGB1A1 [Homo sapiens] | 7356 | SCGB1A1 |
| | | | sp6, T7 | DH10B | | | | |
| 194 | Cam | pOTB7 | −21M13, M13 reverse, | GeneHogs | 3624 | INHBA [Homo sapiens] | 3624 | INHBA |
| | | | sp6, T7 | DH10B | | | | |
| 195 | Cam | pDNR- | −21M13 | GeneHogs | 8835 | SOCS2 [Homo sapiens] | 8835 | SOCS2 |
| | | LIB | | DH10B | | | | |
| 196 | Cam | pOTB7 | −21M13, M13 reverse, | GeneHogs | 9255 | SCYE1 [Homo sapiens] | 9255 | SCYE1 |
| | | | sp6, T7 | DH10B | | | | |
| 197 | Cam | pOTB7 | −21M13, M13 reverse, | GeneHogs | 1435 | CSF1 [Homo sapiens] | 1435 | CSF1 |
| | | | sp6, T7 | DH10B | | | | |
| 198 | Cam | pDNR- | −21M13 | GeneHogs | 6356 | CCL11 [Homo sapiens] | 6356 | CCL11 |
| | | LIB | | DH10B | | | | |
| 199 | Cam | pDNR- | −21M13 | GeneHogs | 9573 | GDF3 [Homo sapiens] | 9573 | GDF3 |
| | | LIB | | DH10B | | | | |
| 200 | Cam | pDNR- | −21M13 | DH10B TonA | 6375 | XCL1 [Homo sapiens] | 6375 | XCL1 |
| | | LIB | | | | | | |
| 201 | Cam | pOTB7 | −21M13, M13 reverse, | GeneHogs | 8741 | INFSF13 [Homo sapiens] | 8742 | TNFSF12 |
| | | | sp6, T7 | DH10B | | | | |
| 202 | Cam | pDNR- | −21M13 | GeneHogs | 5764 | PTN [Homo sapiens] | 5764 | PTN |
| | | LIB | | DH10B | | | | |
| 203 | Cam | pOTB7 | −21M13, M13 reverse, | GeneHogs | 5008 | OSM [Homo sapiens] | 5008 | OSM |
| | | | sp6, T7 | DH10B | | | | |
| 204 | Cam | pOTB7 | −21M13, M13 reverse, | GeneHogs | 6348 | CCL3 [Homo sapiens] | 6348 | CCL3 |
| | | | sp6, T7 | DH10B | | | | |
| 205 | Cam | pDNR- | −21M13 | GeneHogs | 10673 | TNFSF13B [Homo sapiens] | 10673 | TNFSF13B |
| | | LIB | | DH10B | | | | |
| 206 | Cam | pDNR- | −21M13 | DH10B TonA | 3535, 3576 | IGL@ [Homo sapiens], | 3535 | IGL@ |
| | | LIB | | | | IL8 [Homo sapiens] | | |
| 207 | Cam | pOTB7 | −21M13, M13 reverse, | DH10B TonA | 3550 | IK [Homo sapiens] | 3550 | IK |
| | | | sp6, T7 | | | | | |
| 208 | Cam | pOTB7 | −21M13, M13 reverse, | DH10B TonA | 200081 | TXLNA [Homo sapiens] | 200081 | TXLNA |
| | | | sp6, T7 | | | | | |
| 209 | Cam | pOTB7 | −21M13, M13 reverse, | GeneHogs | 537 | ATP6AP1 [Homo sapiens] | 537 | ATP6AP1 |
| | | | sp6, T7 | DH10B | | | | |
| 210 | Cam | pOTB7 | −21M13, M13 reverse, | GeneHogs | 1020 | CDK5 [Homo sapiens] | 1020 | CDK5 |
| | | | sp6, T7 | DH10B | | | | |

TABLE 7

| | | | | | Entrez | | NCBI. | NCBI. |
|---|---|---|---|---|---|---|---|---|
| | Antibiotic | Vector | Sequence | Host | Gene | Gene | GeneID | GeneSymbol |
| NO | Resistance | Name | Primer | Name | List | Symbol | List | List |
| 211 | Kan | pCR-BluntII- | M13(−21), M13 reverse, T7, | DH10B | 651 | BMP3 [Homo sapiens] | 651 | BMP3 |
| | | TOPO | sp6 | TonA | | | | |
| 212 | Kan | pCR-BluntII- | M13(−21), M13 reverse, T7, | DH10B | 27190 | IL17B [Homo sapiens] | 27190 | IL17B |
| | | TOPO | sp6 | TonA | | | | |
| 213 | Kan | pCR-BluntII- | M13(−21), M13 reverse, T7, | DH10B | 27179 | IL1F6 [Homo sapiens] | 27179 | IL1F6 |
| | | TOPO | sp6 | TonA | | | | |
| 214 | Kan | pCR-BluntII- | M13(−21), M13 reverse, T7, | DH10B | 3586 | IL10 [Homo sapiens] | 3586 | IL10 |
| | | TOPO | sp6 | TonA | | | | |
| 215 | Kan | pCR-BluntII- | M13(−21), M13 reverse, T7, | DH10B | 84639 | IL1F10 [Homo sapiens] | 84639 | IL1F10 |
| | | TOPO | sp6 | TonA | | | | |
| 216 | Kan | pCR-XL-TOPO | M13 (−21), M13 reverse, T7 | DH10B | 727 | C5 [Homo sapiens] | 727 | C5 |
| | | | | TonA | | | | |
| 217 | Kan | pCR-BluntII- | M13(−21), M13 reverse, T7, | DH10B | 55914 | ERBB2IP [Homo | 55914 | ERBB2IP |
| | | TOPO | sp6 | TonA | | sapiens] | | |
| 218 | Kan | pCR-BluntII- | M13(−21), M13 reverse, T7, | DH10B | 3451 | IFNA17 [Homo sapiens] | 3451 | IFNA17 |
| | | TOPO | sp6 | TonA | | | | |
| 219 | Kan | pCR-BluntII- | M13(−21), M13 reverse, T7, | DH10B | 338376 | IFNE1 [Homo sapiens] | 338376 | IFNE1 |
| | | TOPO | sp6 | TonA | | | | |
| 220 | Kan | pCR-BluntII- | M13(−21), M13 reverse, T7, | DH10B | 3456 | IFNB1 [Homo sapiens] | 3456 | IFNB1 |
| | | TOPO | sp6 | TonA | | | | |
| 221 | Kan | pCR-BluntII- | M13(−21), M13 reverse, T7, | DH10B | 56300 | IL1F9 [Homo sapiens] | 56300 | IL1F9 |
| | | TOPO | sp6 | TonA | | | | |
| 222 | Kan | pCR-BluntII- | M13(−21), M13 reverse, T7, | DH10B | 6360 | CCL16 [Homo sapiens] | 6360 | CCL16 |
| | | TOPO | sp6 | TonA | | | | |
| 223 | Kan | pCR-BluntII- | M13(−21), M13 reverse, T7, | DH10B | 2661 | GDF9 [Homo sapiens] | 2661 | GDF9 |
| | | TOPO | sp6 | TonA | | | | |
| 224 | Kan | pCR-BluntII- | M13(−21), M13 reverse, T7, | DH10B | 3596 | IL13 [Homo sapiens] | 3596 | IL13 |
| | | TOPO | sp6 | TonA | | | | |
| 225 | Amp | pDrive | T7, sp6 | E. coli | 7124 | TNF [Homo sapiens] | 7124 | TNF |
| 226 | Amp | pINCY | −21M13, M13 reverse, T7, | E. coli | 6369 | CCL24 [Homo sapiens] | 6369 | CCL24 |
| | | | sp6 | | | | | |
| 227 | Amp | pCR4-TOPO | M13 (−21), M13 reverse, T7, | DH10B | 3716 | JAK1 [Homo sapiens] | 3716 | JAK1 |
| | | | T3 | TonA | | | | |

1-2. Packaging and Transfection of Lentivirus

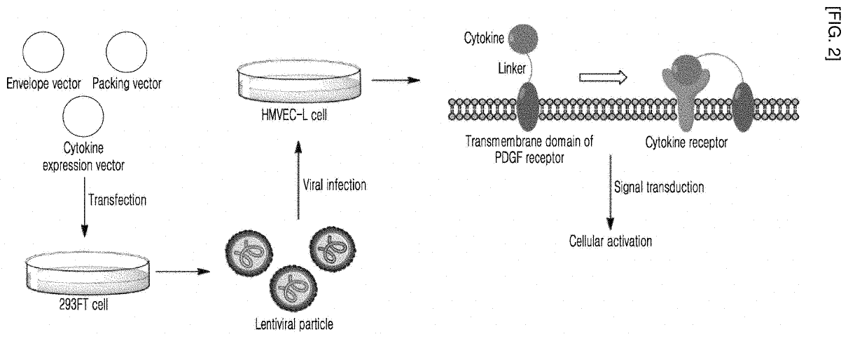

FIG. 2 shows a diagram schematically illustrating a cytokine screening process in a cell after transfecting a cell with a fusion polypeptide according to an example. HEK-293FT cells were simultaneously transfected with the lentiviral plasmid prepared in Example 1-1 together with pCMVD8.9 and pVSVg virus packaging vectors, at a ratio of 1:1:1. After the cells were incubated overnight, the DNA lipid complex was removed, and fresh medium was added to the cells. After 48 hours therefrom, the supernatant containing virus was collected and the collected supernatant was filtered using a 0.22-μm polyether sulfone membrane filter unit (Millipore). The lentiviral particles obtained therefrom were added to human microvascular endothelial cells (HMVEC-L) in a growth medium containing 5 μg/mL of polybrene and was incubated at 37° C. in 5% CO₂ conditions for 24 hours. The medium was purchased from Lonza (Basel, Switzerland) and used.

1-3. Statistical Analysis

Statistical analysis was performed using ANOVA test, two-way ANOVA test, nonparametric T-test, and Mann-Whitney test and experimental data were expressed as mean±standard deviation (SD). If P value<0.05, it was considered significant.

### Example 2: Screening for Functional Cytokines Using Cytokine Library

As shown in FIG. 2, in the cytokine library according to an example, the cytokine is immobilized on the plasma membrane of the target cell by the transmembrane domain to be displayed on the surface of the cell. In other words, since the display of cytokines bound to the plasma membrane reduces receptor-mediated endocytosis in cells and increases the effective molar concentration for the target receptor, the effect of cytokines on target cells can be improved and it may enable continuous cell stimulation by cytokines.

Accordingly, this Example was intended to experimentally confirm the above-mentioned effect by screening cytokines capable of inhibiting vascular endothelial growth factor (VEGF)-dependent proliferation of HMVEC-L using the cytokine library of Example 1. Each lentivirus to the interleukin family (IL-1, IL-2, IL-3, IL-4, IL-6, IL-7, IL-8, IL-9, IL-10, IL-11, IL-12, IL-15, IL-16, IL-17, IL-18, IL-19, IL-20, IL-22, IL-23, IL-24, IL-25, IL-27 and IL-28) was independently transfected with HMVEC-L. The lentivirus was independently transfected with HMVEC-L. Thereafter, the effect of the cytokine on the VEGF-dependent proliferation of HMVEC-L was confirmed by calculating the fold change for the proliferation of untransfected HMVEC-L.

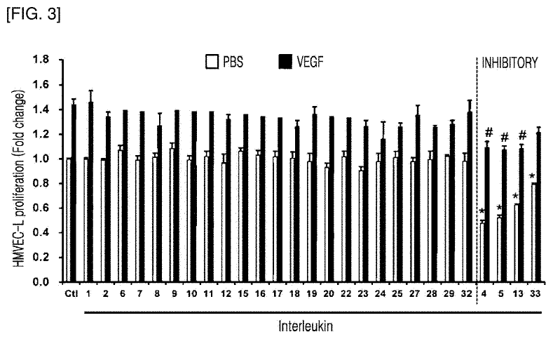

FIG. 3 shows a result of screening cytokines affecting VEGF-dependent proliferation of HMVEC-L using the cytokine library according to an example. As shown in FIG. 3, IL-4, IL-5, IL-13 and IL-33 as cytokines that inhibit VEGF-dependent proliferation of HMVEC-L were identified. The inhibitory effect of IL-4, IL-13 and IL-33 on vascular endothelial cell proliferation has been previously reported through a number of literatures, so these results demonstrate the effectiveness of the screening according to this Example and this cytokine library indicates that it can be used to screen for changes in biological function by the cytokine treatment. Furthermore, hereinafter, the effectiveness of the screening according to this Example was re-verified by confirming the anti-angiogenic effect of IL-5, which showed the strongest effect among cytokines that inhibit VEGF-dependent proliferation of HMVEC-L.

### Example 3: Validation of Screening for Functional Cytokines

In this Example, the anti-angiogenic effect of IL-5 was confirmed through the proliferative ability, migration ability and tube formation efficacy of vascular endothelial cells and the effectiveness of the screening according to this Example was confirmed by confirming specific mechanisms thereof.

3-1. Changes in Proliferative Ability of Vascular Endothelial Cells

The proliferation of HMVEC-L was evaluated by MTT analysis using Cell Titer 96 Aqueous One Solution Cell Proliferation Assay (Promega, USA). Specifically, recombinant human IL-5 and VEGF (type A, 10 ng/ml) (R&D Systems) at various concentrations (0.1, 0.5, 1 or 10 ng/ml) was treated in 96-plate wells containing 2000 HMVEC-Ls and incubated for 72 hours and then the proliferation of HMVEC-L was evaluated at a wavelength of 490 nm using a microplate reader (Bio-Rad, USA). In addition, 5 ng/ml of IL-5 and 10 ng/ml of VEGF were treated in 96-plate wells containing HMVEC-L and incubated for 1 to 5 days. Thereafter, 100 μl of 5% MTS was added to a 96-well plate and incubated for 2 hours and then the proliferation of HMVEC-L was evaluated in the same manner as above. On the other hand, the control group was set to the group treated with IL-5 and PBS.

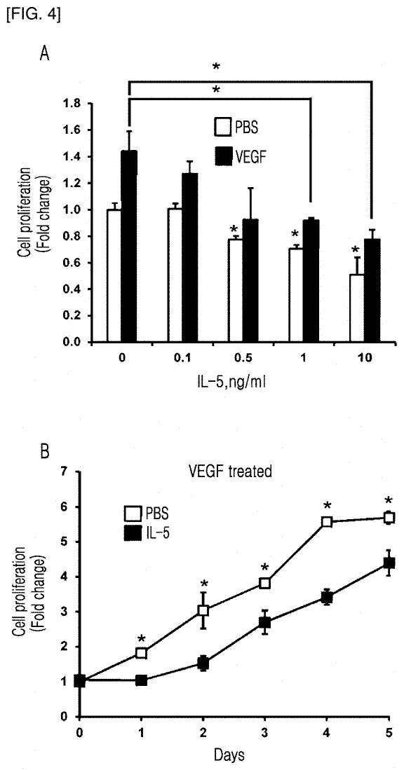

FIG. 4 shows a result of confirming the effect of IL-5 on the proliferation of vascular endothelial cells induced by VEGF through MTT analysis. As shown in FIG. 4A, IL-5 inhibited the basal proliferation of HMVEC-Ls in a concentration-dependent manner.

Moreover, even when IL-5 and VEGF were treated together, the same effect as described above was observed from the time when the treatment concentration of IL-5 was 1 ng/ml or more, which indicated that IL-5 inhibited the proliferation of vascular endothelial cells induced by VEGF of HMVEC-Ls. In addition, as shown in FIG. 4B, as the treatment time of IL-5 increased, the proliferation rate of vascular endothelial cells induced by VEGF was significantly decreased.

3-2. Changes in Migration Ability of Vascular Endothelial Cells

The migration ability of HMVEC-L was evaluated by wound-healing assay. Specifically, HMVEC-L was seeded on a 24-well culture plate. Then, a recoverable wound was induced on HMVEC-L using CytoSelect™ 24-Well Wound Healing Assay kit (Cell Biolabs, INC). Thereafter, IL-5 and/or VEGF were added thereto, and after incubation for 18 hours, closure of the wound was observed through the Image J software module. The closure rate was calculated by the following equation.

Closure rate (%)=W₀−Wₙ/W₀×100 [Equation]

(W₀: width of wound at 0 hours, Wₙ: width of wound at n hours)

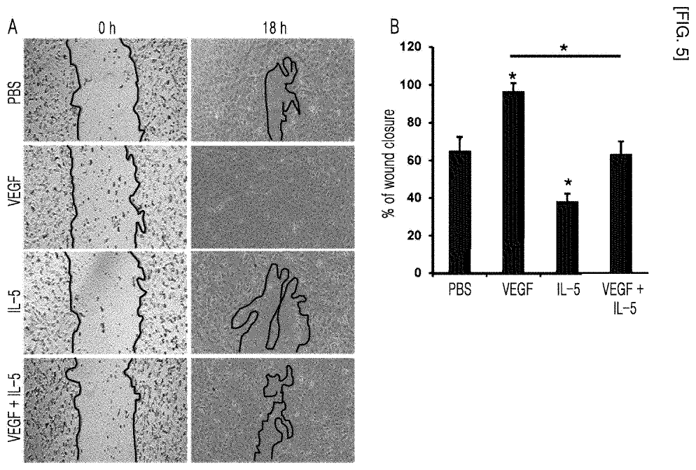

FIG. 5 shows a result of confirming the effect of IL-5 on the migration ability of vascular endothelial cells induced by VEGF through wound-healing analysis. As shown in FIG. 5, the treatment of IL-5 for HMVEC-L reduced the wound closure rate by about 27% compared to the PBS-treated control group, and even in the condition treated with VEGF, IL-5 inhibited wound healing by about 33% compared to the group treated with VEGF alone.

3-3. Change in Tube Formation Ability of Vascular Endothelial Cell

The tube formation ability of HMVEC-L was evaluated by a conventional tube formation assay. Specifically, HMVEC-Ls of 10000 were incubated with 5 ng/ml IL-5 and/or 10 ng/ml VEGF in a matrigel in a u-plate angiogenic 96-well plate (Ibidi) in (Standard Formulation, BD Biosciences) for 25 hours. Thereafter, the total number of branch points and the total length of the formed tube were calculated through Image J analysis software.

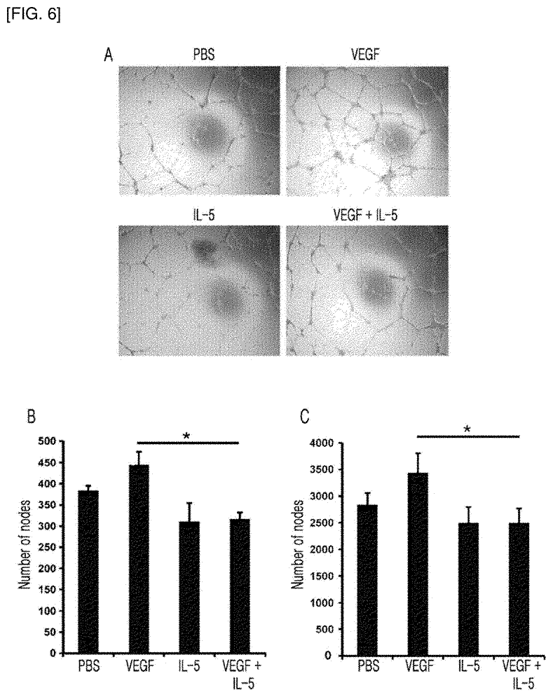

FIG. 6 shows a result of confirming the effect of IL-5 on the tube formation ability of vascular endothelial cells induced by VEGF through tube formation assay. As shown in FIG. 6, in the condition treated with VEGF, the treatment with IL-5 significantly reduced the total number of branch points and the total length of formed tubes compared to the group treated with VEGF alone.

3-4. Identification of Mechanism of Anti-Angiogenesis

In order to confirm the intracellular signaling mechanism of anti-angiogenesis by treatment with IL-5, STAT5 expression of HMVEC-L was knocked down, and the corresponding anti-angiogenic effect was experimentally confirmed. Specifically, STAT5 expression of HMVEC-L was knocked down by ON-TARGET and SMARTpool STAT siRNA (Dharmacon). Transfection of endothelial cell monolayers was carried out by incubating 300,000 cells per well for 6 hours with siRNA at final concentration of 50 nM in Oligofectamine and serum-free Opti-MEM (Invitrogen). After incubation with siRNA/Oligofectamine mix for 6 hours, HMVEC-L was washed and 2 ml of complete ECGM medium was added to each well. After transfection, cells were transferred to 96 well plates for functional analysis.

Meanwhile, for Western blot analysis, the HMVEC-L-derived lysate was denatured in Laemmli sample buffer (95° C. for 5 minutes), separated by SDS-PAGE, and then transferred to a nitrocellulose membrane. The nitrocellulose membrane was blocked in PBST containing 5% BSA for 1 hour, and then incubated overnight at 4° C. with the primary antibody. After washing the nitrocellulose membrane several times with PBST, the blot was incubated with HRP (horseradish peroxidase)-conjugated anti-human antibody or anti-rabbit antibody for 1 hour. Subsequently, the nitrocellulose membrane was washed with PBST and developed with ECL. Anti IL-5RA, anti IL-5RB, STAT1, STAT5, pSTAT1 and pSTAT5 antibodies were obtained from Cell Signaling Technology.

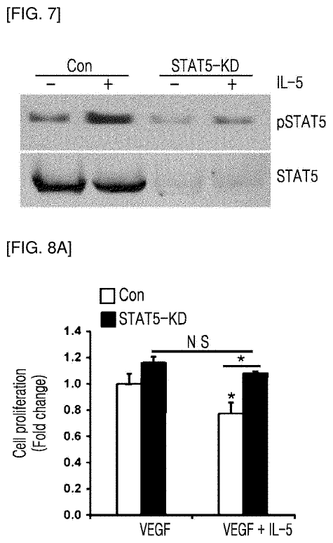

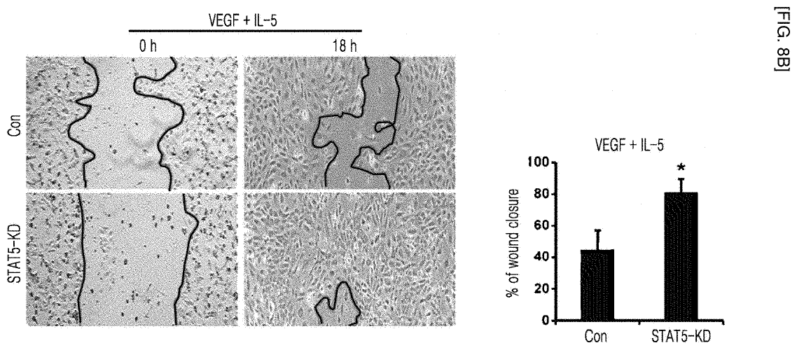

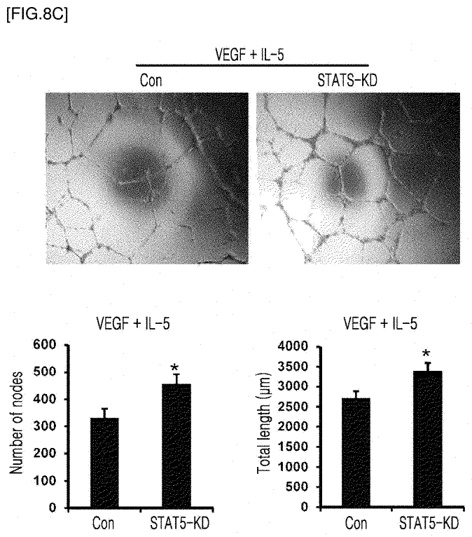

FIG. 7 shows a result of confirming the phosphorylation of STAT5 by IL-treatment through Western blotting, and FIG. 8A to FIG. 8C show results confirming the effect of treatment of IL-5 on the anti-angiogenic effect induced by VEGF for HMVEC-L in which STAT5 was knocked down. As shown in FIG. 7, IL-5 treatment strongly induced the phosphorylation of STAT5 in HMVEC-Ls. In addition, as shown in FIG. 8A to FIG. 8C, knock-down of STAT5 offset the inhibitory effect of the proliferation, migration and tube formation abilities of IL-5 on HMVEC-Ls. This indicates that the activation of STAT5 mediates the anti-angiogenic effect of IL-5 on vascular endothelial cells.

These series of experimental results suggest that IL-5 can exhibit an anti-angiogenic effect on vascular endothelial cells even in the presence of a strong pre-angiogenic factor, VEGF which are the result of verifying the effectiveness of screening according to this Example.

1. A fusion polypeptide comprising:
cytokines;
transmembrane domain; and
a linker connecting the cytokine and the transmembrane domain.

2. The fusion polypeptide of claim 1, wherein the transmembrane domain is a transmembrane domain of receptor tyrosine kinases (RTKs).

3. The fusion polypeptide of claim 2, wherein the transmembrane domain of the receptor tyrosine kinases is any one selected from the group consisting of epidermal growth factor receptor, insulin receptor, platelet-derived growth factor receptor, vascular endothelial growth factor receptor, fibroblast growth factor receptor, cholecystokinin (CCK) receptor, neurotrophic factor (NGF) receptor, hepatocyte growth factor (HGF) receptor, ephrin (Eph) receptor, angiopoietin receptor and RYK (related to receptor tyrosine kinase).

4. The fusion polypeptide of claim 1, wherein the cytokine is any one selected from the group consisting of BMP (bone morphogenetic protein) family, CCL (cheomkine ligands) family, CMTM (CKLF-like MARVEL transmembrane domain containing member) family, CXCL (C-X-C motif ligand ligand) family, GDF (growth/differentiation factor) family, growth hormone, IFN (Interferon) family, IL (Interleukin) family, TNF (tumor necrosis factors) family and a combination thereof.

5. The fusion polypeptide of claim 1, wherein the cytokine is any one selected from the group consisting of REG, ATP6AP1, BMP10, BMP15, BMP2, BMP3, BMP4, BMP5, BMP7, BMP8A, BMP8B, BRE, C5, CCL1, CCL8, CCL11, CCL13, CCL14, CCL16, CCL17, CCL18, CCL19, CCL2, CCL20, CCL21, CCL22, CCL23, CCL24, CCL25, CCL26, CCL28, CCL3, CCL3L1, CCL3L3, CCL4, CCL4L1, CCL4L2, CCL5, CCL7, CD40LG, CD70, CDKS, CER1, CKLF, CLCF1, CMTM1, CMTM2, CMTM3, CMTM4, CMTM5, CMTM6, CMTM7, CMTM8, CNTF, CSF1, CSF2, CSF3, CSH1, CSH2, CTF1, CX3CL1, CXCL1, CXCL10, CXCL11, CXCL12, CXCL13, CXCL14, CXCL16, CXCL2, CXCL3, CXCLS, CXCL6, CXCL9, CYP26B1, EBI3, EDA, EPO, ERAP1, ERBB2IP, FAM3B, FAM3C, FAM3D, FASLG, FGF10, FGF12, FIGF, FLT3LG, GDF10, GDF15, GDF2, GDF3, GDFS, GDF9, GH1, GLMN, GPI, GREM1, GREM2, GRN, IFNA1, IFNA10, IFNA13, IFNA14, IFNA17, IFNA2, IFNA21, IFNA4, IFNA5, IFNA6, IFNA7, IFNA8, IFNB1, IFNE1, IFNG, IFNW1, IGL1, IK, IL10, IL11, IL12A, IL12B, IL13, IL15, IL16, IL17A, IL17B, IL17C, IL17D, IL17F, IL18, IL19, ILIA, IL1B, IL1F10, IL1F5, IL1F6, IL1F7, IL1F8, IL1F9, IL1RN, IL2, IL20, IL21, IL22, IL23A, IL24, IL25, IL27, IL28A, IL28B, IL29, IL3, IL32, IL33, IL4, IL5, IL6, IL7, IL8, IL9, INHA, INHBA, INHBB, JAK1, KITLG, LASS1, LEFTY1, LEFTY2, LIF, LTA, LTB, MDK, MIF, MSTN, NAMPT, NODAL, NRG1, OSM, PDGFA, PDGFB, PF4, PF4V1, PIK3R1, PPBP, PRL, PTEN, PTN, PXMP2, RHOQ, SCG2, SCGB1A1, SCGB3A1, SCG2, SCYE1, SDCBP, SECTM1, SIVA1, SLCO1A2, SLURP1, SOCS2, SPP1, SPRED2, THPO, TNF, TNFRSF11B, TNFSF10, TNFSF11, TNFSF12, TNFSF13, TNFSF13B, TNFSF14, TNFSF15, TNFSF18, TNFSF8, TNFSF9, TRADD, TRAP1, TRIP6, TSLP, TXLNA, TYMP, VEGFA, VEGFB, VEGFC, WNT16, XCL1, XCL2, YARS and combinations thereof.

6. The fusion polypeptide of claim 1, wherein the cytokine is autocrine to a target cell.

7. The fusion polypeptide of claim 1, wherein the transmembrane domain is immobilized by penetrating a cell membrane of a target cell, and the cytokine binds to cell membrane receptor of the target cell to stimulate the target cell.

8. The fusion polypeptide of claim 1, wherein the linker is a flexible linker and consists of 1 to 400 amino acid residues.

9. The fusion polypeptide of claim 1, wherein the linker is (GGGGS)ₙ (SEQ ID NO: 1), (SGGGG)ₙ (SEQ ID NO: 2), (SRSSG)ₙ (SEQ ID NO: 3), (SGSSC)ₙ (SEQ ID NO: 4), (GKSSGSGSESKS)ₙ (SEQ ID NO: 5), (RPPPPC)ₙ (SEQ ID NO: 6), (SSPPPPC)ₙ (SEQ ID NO: 7), (GSTSGSGKSSEGKG)ₙ (SEQ ID NO: 8), (GSTSGSGKSSEGSGSTKG)ₙ (SEQ ID NO: 9), (GSTSGSGKPGSGEGSTKG)ₙ (SEQ ID NO: 10), or (EGKSSGSGSESKEF)ₙ (SEQ ID NO: 11), wherein n is an integer of 1 to 20.

10. A polynucleotide encoding the fusion polypeptide of claim 1.

11. A vector comprising the polynucleotide of claim 10.

12. The vector of claim 11, wherein the vector is selected from the group consisting of a plasmid, a lentivirus, an adenovirus, an adeno-related virus, a retrovirus, a herpes simplex virus and a vaccinia virus.

13. A host cell comprising the vector of claim 11.

14. The host cell of claim 13, wherein the host cell is an animal cell or a plant cell.

15. A cytokine library comprising a plurality of the fusion polypeptides of claim 1, the polynucleotides of claim 10, the vector of claim 11, or the host cell of claim 13.

16. A method of screening cytokine comprising identifying any one change selected from the group consisting of a biological change of cell by a cytokine from the host cell of claim 10, a change in the expression or activity of an exogenous or endogenous gene or protein and a combination thereof.
